(19) United States
(12) Patent Application Publication
(10) Pub. No.: US 2004/0254471 A1
(43) Pub. Date: Dec. 16, 2004

(54) Miniature ultrasonic phased array for intracardiac and intracavity applications

(76) Inventors: Andreas Hadjicostis, Carmel, IN (US); Jared Brosch, Cicero, IN (US); Patrick Gwin, Indianapolis, IN (US)

Correspondence Address: Woodard, Emhardt, Moriarty, McNett & Henry LLP, Bank One Center/Tower, 111 Monument Circle, Suite 3700, Indianapolis, IN, 46204-5137, US

(21) Appl. No.: 10/686,120

(22) Filed: Oct. 15, 2003

Related U.S. Application Data
(60) Provisional application No. 60/478,649, filed on Jun. 13, 2003.

(51) Int. Cl. A61B 8/14

(52) U.S. Cl. 600/459

(57) ABSTRACT

One embodiment of the present application includes providing an array ultrasound imaging phase assembly that includes a substrate and several piezoelectric elements each mounted on the substrate. The elements are spaced apart from each other and arranged in a noncylindrical pattern. An acoustic backing block is mounted to the substrate on a side opposite its matching layers. The array can be positioned along a desired region within a subject's body by movement through the circulatory system or other cavities. While the array assembly is positioned along this region, ultrasonic interrogation of an internal portion of the subject's body can be performed to generate one or more images.

### CROSS-REFERENCE TO RELATED APPLICATIONS

The present application claims the benefit of U.S. Provisional Patent Application No. 60/478,649 filed 13 Jun. 2003, which is hereby incorporated by reference. The present application is related to the commonly owned U.S. Patent Application entitled: “MULTI-ELEMENT ARRAY FOR ACOUSTIC ABLATION”invented by Brosch et al. and filed on even date herewith, and the commonly owned U.S. Patent Application entitled: “COMPOSITIONS FOR HIGH POWER PIEZOELECTRIC CERAMICS” invented by Liufu and filed on even date herewith, all of which are hereby incorporated by reference.

### BACKGROUND

The present invention relates to multi element arrays for ultrasound applications, and more particularly, but not exclusively, relates to the fabrication, use, and structure of devices including an array of elements to generate ultrasonic energy for medical use.

Heart disease represents one of the most common debilitating diseases among the elderly, and is a common cause of death. The mammalian heart typically has four chambers: two ventricles for pumping the blood and two atria, each for collecting the blood from the vein leading to it and delivering that blood to the corresponding ventricle. The left ventricle pumps blood to the vast bulk of the mammalian body. As a result, problems with the left ventricle or with the mitral valve, which leads from the left atrium into the left ventricle, can cause serious health problems. When it appears that a patient has inadequate blood circulation in a portion of his or her body, the left ventricle and the mitral valve are often suspect. Specifically diagnosing a problem with these structures; however, is not always an easy proposition. In fact, unnecessary surgeries are sometimes performed due to the difficulty of forming a proper diagnosis.

More particularly, cardiac arrhythmia—especially atrial fibrillation—persist as common and dangerous medical aliments associated with abnormal cardiac chamber wall tissue. In patients with cardiac arrhythmia, abnormal regions of cardiac tissue do not follow the synchronous beating cycle associated with normally conductive tissue in patients with sinus rhythm. Instead, the abnormal regions of tissue aberrantly conduct to adjacent tissues, which disrupts the cardiac cycle causing an asynchronous rhythm. Such abnormal conduction is known to occur at various regions of the heart.

Irregular cardiac function and corresponding hemodynamic abnormalities caused by atrial fibrillation in particular can result in stroke, heart failure, and other medical problems. In fact, atrial fibrillation is believed to be a significant cause of cerebral stroke, wherein the hemodynamic abnormality in the left atrium caused by the fibrillatory wall motion precipitate the formation of thrombus within the atrial chamber. A thromboembolism is ultimately dislodged into the left ventricle which thereafter pumps the embolism into the cerebral circulation resulting in a stroke. Accordingly, numerous procedures for treating atrial arrhythmias have been developed, including pharmacological, surgical, and catheter ablation procedures.

Among these, the less invasive catheter-based approaches have generally been targeted to atrial segmentation with ablation catheter devices adapted to form linear or curvilinear electrophysiologic lesions in the atrial wall or pulmonary vein to disrupt aberrant atrial node signaling through the tissue. Unfortunately, currently available methods for imaging the heart and specifically the left ventricle and the mitral valve leave much to be desired. One scheme, termed transthoracic imaging, typically requires the placement of an ultrasound transceiver against the chest of the patient and the use of this transceiver to image the heart. One drawback of this scheme is that the bones and the other tissue types that are interposed between the ultrasound transceiver and the heart during this procedure prevent the formation of a sufficiently detailed image of the heart. Another cardiac imaging method, transesophageal imaging, involves the insertion of an ultrasound transceiver into the esophagus of the patient. Although transesophageal imaging places the ultrasound transceiver closer to the heart, the patient must be rendered unconscious by way of a general anesthetic for this method to be employed. For cardiac imaging, it can be highly desirable to have a conscious, responsive patient who can change position upon request in order to facilitate the imaging of the heart under various conditions.

Accordingly, there is an interest in techniques, devices, and systems for intracardiac and/or intravascular imaging with ultrasound. Unfortunately, providing an ultrasonic imaging probe of a desired resolution that is suitable for use within the heart and/or vasculature is often difficult. Thus, further contributions in this area of technology are desired.

### SUMMARY

One embodiment of the present invention is a unique ultrasonic imaging multi-element array. Other embodiments include unique methods, systems, devices, and apparatus for generating and/or detecting ultrasound. As used herein, “ultrasound” and “ultrasonic” refer to acoustic energy waveforms having a frequency of more than 20,000 Hertz (Hz) through one or more media at standard temperature and pressure.

A further embodiment of the present invention includes an array comprised of several piezoelectric elements and cabling comprised of several electrical conductors each insulated from one another. The conductors are each electrically connected to a different one of the elements to provide an independent electrical signal pathway for each. In one form, the array includes a substrate with a number of electrically conductive traces. The elements are mounted to the substrate so that each makes electrical contact with a different one of the traces. These traces are each electrically coupled to a different one of the conductors of the cabling. A backing layer can be mounted to a side of the substrate opposite the side to which the elements are mounted. The array and cabling can be structured to place the array at a desired intracardiac site through a circulatory system of a human subject while a proximal end portion of the cabling remains outside the human subject. In one particular form, the array has a maximum cross-sectional dimension of 4 millimeters (mm) or less taken perpendicular to a longitudinal centerline of the array. Preferably, the array includes at least 24 elements. More preferably, the array includes at least 32 elements. Even more preferably, the array includes 48 elements or more.

Another embodiment of the present invention includes: providing a piezoelectric work piece mounted to a first side of a substrate that carries a number of electrically conductive traces; dividing the piezoelectric work piece into 24 or more elements with each electrically coupling to a different one of the traces; and attaching a backing layer to a second side of the substrate opposite the first side to which the work piece was mounted. The backing layer is operable to augment the bandwidth of the device during ultrasonic operation, and to reduce undesired acoustic reflection, being typically comprised of an ultrasound-absorbing material. In one form, this embodiment further includes masking the work piece and substrate before dividing it to expose a surface of the work piece and an electrically conductive pad carried by the substrate. An electrically conductive material is then deposited to electrically couple the exposed surface and pad before the work piece is divided. Alternatively or additionally, cabling is attached to the substrate that includes 24 or more conductors each electrically insulated from one another and each electrically connected to a different one of the traces.

Still another embodiment includes an elongate device having a proximal end portion and a distal end portion. The device has an array of 24 or more piezoelectric elements mounted to a substrate to electrically make contact with a corresponding one of a number of electrically conductive traces carried by the substrate with the array being located along the distal end portion of the device. The device also includes cabling with 24 or more electrical conductors each insulated from one another and each electrically connected to a different one of the electrically conductive traces to correspondingly define an independently operable electrical signal pathway for each of the elements. The device is structured to place the array at a desired intracardiac site through a circulatory system of a human subject while the proximal end portion remains outside the human subject.

In another embodiment, an apparatus includes an ultrasonic array assembly with a substrate having a first side opposite a second side that carries a number of electrically conductive traces. Several piezoelectric elements are mounted on the first side and each is electrically coupled to a different one of the traces. These elements can be arranged in a linear, noncylindrical pattern that may be generally flat or curved. A maximum cross-sectional dimension of the assembly taken perpendicular to a longitudinal centerline is 4 mm or less for this embodiment.

Yet another embodiment of the present invention includes providing an array coupled to cabling in which the array includes a substrate having a first side with several piezoelectric elements mounted thereto and a second side with a backing layer mounted thereto. This backing layer is comprised of a material operable to provide broad bandwidth and to reduce undesired acoustic reflection. The array is positioned along a desired region within a subject's body by movement through a circulatory system. A proximal portion of the cabling remains outside the subject's body while the array is positioned along the desired region. An internal portion of the subject's body is ultrasonically interrogated with the array to generate one or more images corresponding to the internal portion.

A further embodiment includes: providing an array of at least 24 piezoelectric elements mounted to a substrate to electrically couple to a corresponding one of a number of electrically conductive traces carried by the substrate; positioning the array at a desired site by movement through a circulatory system of a subject's body; transmitting a plurality of electrical stimulus signals to the array at the desired site through a proximal end portion of cabling positioned outside the subjects body; and generating ultrasonic energy with each of the elements in response to the electric stimulus signals. In one form, the cabling includes 24 or more signal conductors electrically insulated from one another that are each electrically connected to a different one of the conductive traces to define independently operable signal pathways for each of the elements. Alternatively or additionally, the electric stimulus signals can be provided with equipment coupled to the proximal end portion of the cabling, ultrasound can be detected with one or more of the elements to return a corresponding number of electric response signals to the equipment, and one or more images can be generated with the equipment as a function of the stimulus signals and the response signals.

One object of the present invention is to provide a unique multielement array for ultrasound applications.

Another object of the present invention is to provide a unique method, system, device, or apparatus for generating and/or detecting ultrasound.

Further forms, objects, features, aspects, benefits, advantages, and embodiments of the present invention shall become apparent from the detailed description and drawings provided herewith.

### BRIEF DESCRIPTION OF THE DRAWING

FIG. 1 is a schematic view of a system utilizing ultrasound.

FIG. 2 is a partial cut-away, plan view of an ultrasonic probe device included in the system of FIG. 1.

FIGS. 3 and 4 provide a flowchart illustrating one process for manufacturing the ultrasonic probe device included in the system of FIG. 1.

FIG. 5 is a flowchart illustrating the preparation of a flexible circuit substrate for the process of FIGS. 3 and 4.

FIG. 6 is a flowchart illustrating the preparation of a piezoelectric work piece for the process of FIGS. 3 and 4.

FIG. 7 is a flowchart illustrating a cable connection procedure for the process of FIGS. 3 and 4.

FIG. 8 is a plan view of a first circuit layer for assembly of a circuit substrate included in the ultrasonic probe device manufactured in accordance with the process of FIGS. 3 and 4.

FIG. 9 is a plan view of a second circuit layer for assembly of the circuit substrate included in the ultrasonic probe device manufactured in accordance with the process of FIGS. 3 and 4.

FIG. 10 is a plan view of a third circuit layer for assembly of the circuit substrate included in the ultrasonic probe device manufactured in accordance with the process of FIGS. 3 and 4.

FIG. 11 is a plan view of a partially assembled ultrasonic probe device manufactured in accordance with the process of FIGS. 3 and 4.

FIG. 12 is a partial, cross-sectional view of the partially assembled ultrasonic probe device taken along section line 12—12 of FIG. 11.

FIG. 13 is a partial assembly view of the ultrasonic probe device during cable attachment in accordance with the process of FIGS. 3 and 4.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

One embodiment of the present invention includes an ultrasonic device structured for percutaneous insertion in the human body. The device includes an array of piezoelectric elements located at a distal end portion and cabling connected to the array that extends from the array to a proximal end portion of the device. The elements are carried on a circuit substrate including at least two levels of electrical conductor patterns. The cabling includes multiple conductors each electrically insulated from one another and each electrically connected to a different one of the elements. In one preferred form, the elements number at least 24. In a more preferred form, the elements number at least 32. In a still more preferred form, the elements number at least 48 and are configured to send and receive ultrasound for image generation.

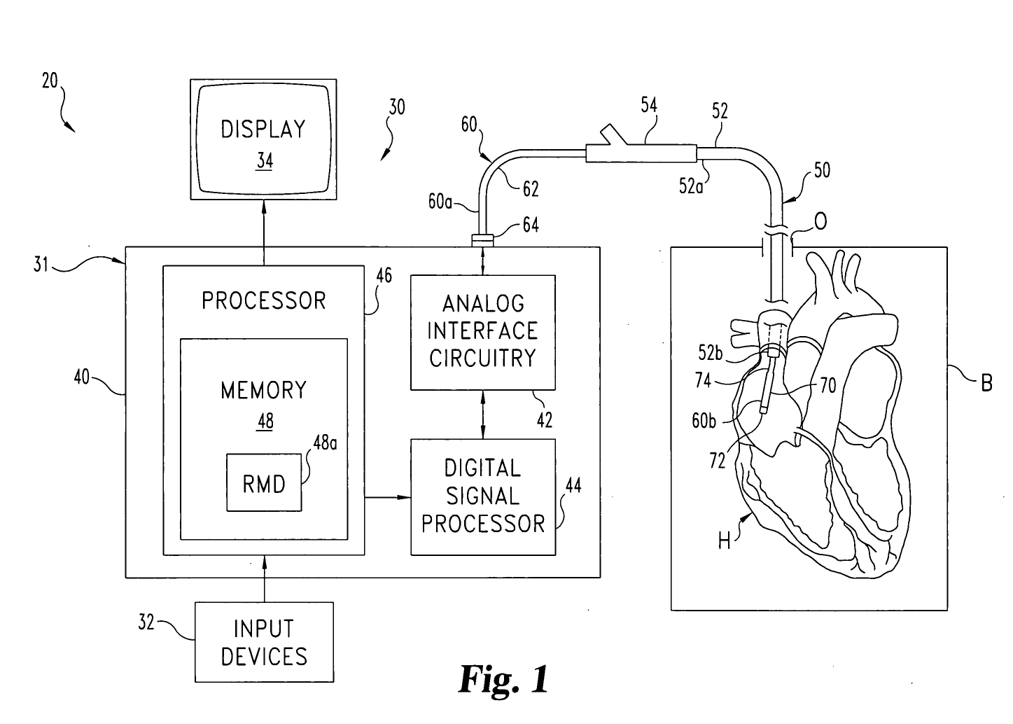

With reference to FIG. 1, further aspects are described in connection with system 20. System 20 is arranged to provide images internal to body B for medical diagnosis and/or medical treatment. System 20 includes control station 30, catheterization equipment 50, and ultrasonic probe device 60. Control station 30 includes equipment 31 coupled to device 60. Device 60 is configured with catheterization equipment 50 for placement within body B of a human patient or subject, as schematically represented in FIG. 1. Equipment 31 includes operator input devices 32 and operator display device 34. Input devices 32 include an alphanumeric keyboard and mouse or other pointing device of a standard variety. Alternatively or additionally, one or more other input devices can be utilized, such as a voice input subsystem or a different type as would occur to those skilled in the art. Operator display device 34 can be of a Cathode Ray Tube (CRT) type, Liquid Crystal Display (LCD) type, plasma type, Organic Light Emitting Diode (OLED) type, or such different type as would occur to those skilled in the art. Alternatively or additionally, one or more other operator output devices can be utilized, such as a printer, one or more loudspeakers, headphones, or such different type as would occur to those skilled in the art. Station 30 also can include one or more communication interfaces suitable for connection to a computer network, such as a Local Area Network (LAN), Municipal Area Network (MAN), and/or Wide Area Network (WAN) like the internet; a medical diagnostic device; another therapeutic device; a medical imaging device; a Personal Digital Assistant (PDA) device; a digital still image or video camera; and/or audio device, to name only a few.

Equipment 31 also includes processing subsystem 40 for processing signals and data associated with system 20. Subsystem 40 includes analog interface circuitry 42, Digital Signal Processor (DSP) 44, data processor 46, and memory 48. Analog interface circuitry 42 is responsive to control signals from DSP 44 to provide corresponding analog stimulus signals to imaging device 60. At least one of analog circuitry 42 and DSP 44 includes one or more digital-to-analog converters (DAC) and one or more analog-to-digital converters (ADC) to facilitate operation of system 20 in the manner to be described in greater detail hereinafter. Processor 46 is coupled to DSP 44 to bidirectionally communicate therewith, selectively provide output to display device 34, and selectively respond to input from operator input devices 32.

DSP 44 and/or processor 46 can be of a programmable type; a dedicated, hardwired state machine; or a combination of these. DSP 44 and processor 46 perform in accordance with operating logic that can be defined by software programming instructions, firmware, dedicated hardware, a combination of these, or in a different manner as would occur to those skilled in the art. For a programmable form of DSP 44 or processor 46, at least a portion of this operating logic can be defined by instructions stored in memory 48. Programming of DSP 44 and/or processor 46 can be of a standard, static type; an adaptive type provided by neural networking, expert-assisted learning, fuzzy logic, or the like; or a combination of these.

Memory 48 is illustrated in association with processor 46; however, memory 48 can be separate from or at least partially included in one or more of DSP 44 and processor 46. Memory 48 includes at least one Removable Memory Device (RMD) 48a. Memory 48 can be of a solid-state variety, electromagnetic variety, optical variety, or a combination of these forms. Furthermore, memory 48 and can be volatile, nonvolatile, or a mixture of these types. Memory 48 can be at least partially integrated with circuitry 42, DSP 44, and/or processor 46. RMD 48a can be a floppy disc, cartridge, or tape form of removable electromagnetic recording media; an optical disc, such as a CD or DVD type; an electrically reprogrammable solid-state type of nonvolatile memory, and/or such different variety as would occur to those skilled in the art. In still other embodiments, RMD 48a is absent.

Circuitry 42, DSP 44, and processor 46 can be comprised of one or more components of any type suitable to operate as described herein. Further, it should be appreciated that all or any portion of circuitry 42, DSP 44, and processor 46 can be integrated together in a common device, and/or provided as multiple processing units. For a multiple processing unit form of DSP 44 or processor 46; distributed, pipelined, and/or parallel processing can be utilized as appropriate. In one embodiment, circuitry 42 is provided as one or more components coupled to a dedicated integrated circuit form of DSP 44; processor 46 is provided in the form of one or more general purpose central processing units that interface with DSP 44 over a standard bus connection; and memory 48 includes dedicated memory circuitry integrated within DSP 44 and processor 46, and one or more external memory components including a removable disk form of RMD 48a. Circuitry 42, DSP 44, and/or processor 46 can include one or more signal filters, limiters, oscillators, format converters (such as DACs or ADCs), power supplies, or other signal operators or conditioners as appropriate to operate system 20 in the manner to be described in greater detail hereinafter.

Equipment 50 includes flexible catheter 52 with proximal end 52a opposite distal end 52b, and catheter port device 54. Proximal end 52a is connected to catheter port device 54 to be in fluid communication therewith. Catheter 52 includes one or more lumens extending therethrough. Equipment 50 is introduced into and removed from body B through opening O in a standard manner that typically includes one or more other components not shown to enhance clarity.

Device 60 has proximal end portion 60a and distal end portion 60b. Device 60 includes electrical cabling 62 with connector 64 electrically connected to equipment 31 of station 30. Cabling 62 extends from connector 64 at proximal end portion 60a through port device 54 and a lumen of catheter 52 to distal end portion 60b. Device 60 includes ultrasonic array assembly 70 and terminates at distal tip 72. Assembly 70 is connected to cabling 62 at distal end portion 60b by interface 74.

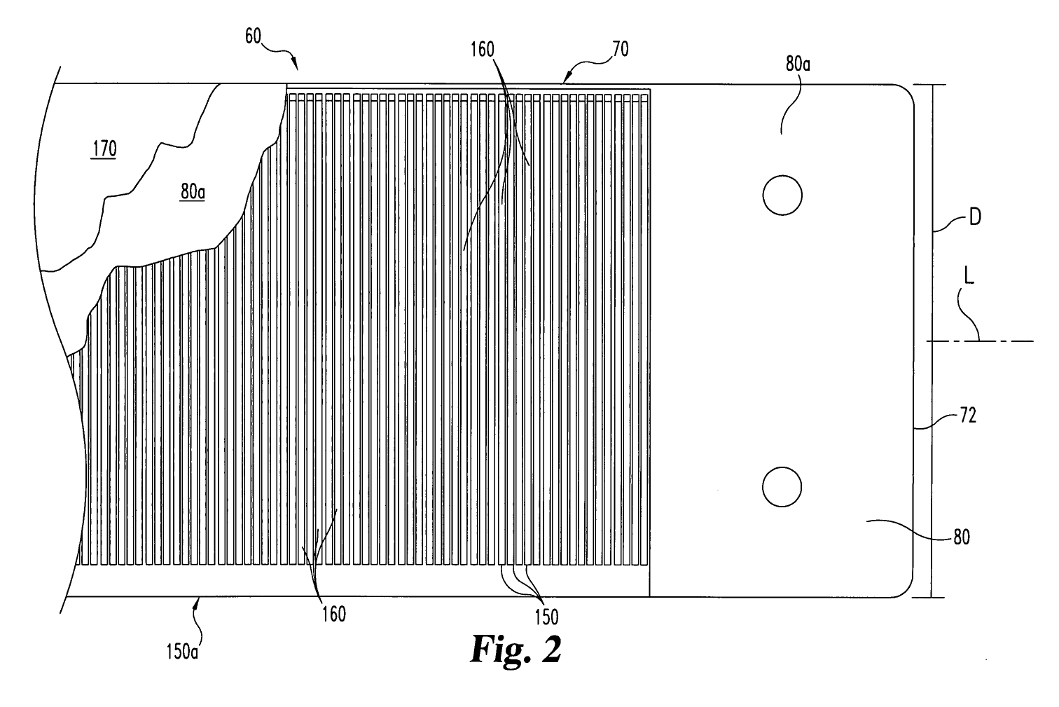

Further aspects of assembly 70 are illustrated in the partial cut away, plan view of FIG. 2. As shown in FIG. 2, assembly 70 extends along an axis corresponding to longitudinal centerline L and includes a multilayer, flexible circuit substrate 80 carrying array 150a. Array 150a includes a number of piezoelectric elements 150 each made of a piezoelectric material that responds to an appropriate electrical stimulus to generate acoustic energy in the ultrasonic frequency range. Elements 150 are elongate with a longitudinal axis approximately perpendicular to centerline L. Elements 150 are each generally sized and shaped the same, and are evenly spaced apart from one another. Elements 150 are generally co-planar—such that each is intersected by a common plane. In one nonlimiting example, such a plane is parallel to the view plane of FIG. 2. Device 60 is depicted with an array dimension D perpendicular to centerline L. For the illustration of FIG. 2, dimension D is in the view plane and corresponds to the maximum cross-sectional dimension of assembly 70 taken perpendicular to centerline L. In contrast, a minimum dimension of assembly 70 perpendicular to centerline L corresponds to assembly thickness and is along a direction generally perpendicular to the view plane of FIG. 2.

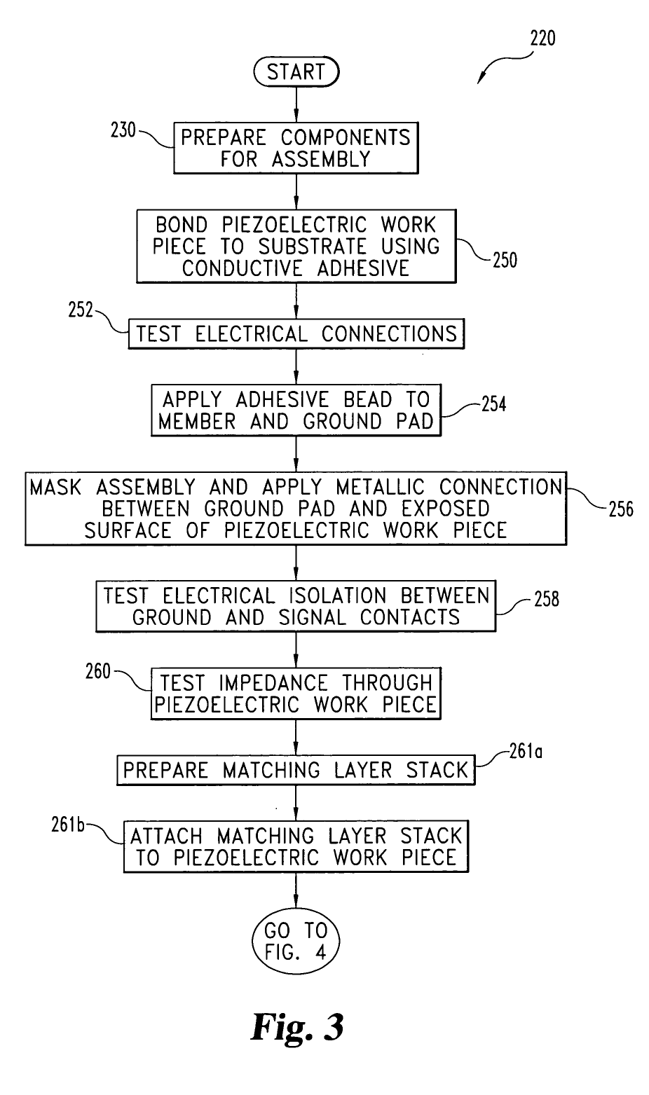

In a preferred embodiment of the present application, elements 150 number 24 or more. In a more preferred embodiment, elements 150 number 32 or more. In an even more preferred embodiment, elements 150 number 48 or more. It should be appreciated that 48 elements 150 are specifically depicted in FIG. 2. Elements 150 can each be made of the same piezoelectric material. Alternatively, one or more elements 150 can be made of a material different than one or more other of elements 150. Certain nonlimiting embodiments for making elements 150 from a piezoelectric ceramic work piece are described in connection with the flow charts of FIGS. 3-7 hereinafter.

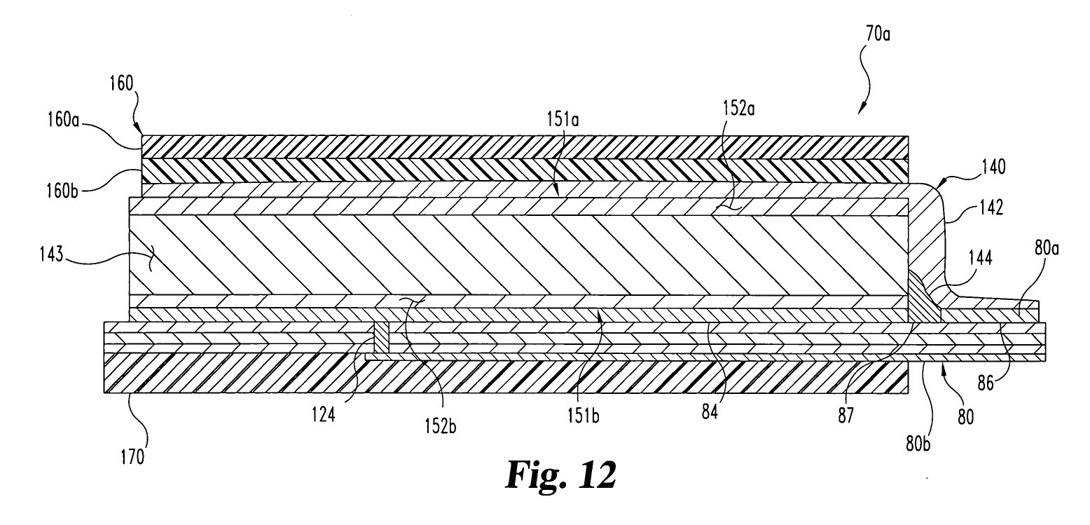

Assembly 70 also includes a number of acoustic matching layer members 160 each mounted to a different corresponding element 150. In one form, each member 160 includes two layers of different acoustic impedance arranged to provide a desired ultrasonic bandwidth. In other forms, members 160 can be arranged with more or fewer layers, may differ from one to the next, or may be absent. It should be understood that only a few of elements 150 and members 160 are designated by reference numerals in FIG. 2 to preserve clarity. Assembly 70 also includes an acoustic backing layer 170 that is mounted to a side of substrate 80 opposite side 80a to which elements 150 are mounted. Because the substrate mounting side for layer 170 is hidden in FIG. 2, layer 170 is illustrated in the successive cut-aways shown in the upper left hand portion of FIG. 2. It should be understood that layer 170 is generally coextensive with elements 150 on the hidden side. Layer 170 is comprised of one or more materials selected to provide broad bandwidth and to reduce, if not eliminate, undesirable acoustic/ultrasonic reflection during operation of device 60. However, in other embodiments layer 170 may not be coextensive with all the elements 150, may differ in relation to one or more elements, or be absent. One form of the acoustic stack of each element 150, corresponding member 160, and layer 170 is further illustrated in connection with the partial, cross-sectional view of FIG. 12 to be further described hereinafter.

In one preferred embodiment, array assembly 70 is shaped and sized with a maximum dimension perpendicular to centerline L of 4 millimeters (mm) or less. In a more preferred embodiment, array assembly 70 is shaped and sized to be used with a 3 to 12 French catheter size. In an even more preferred embodiment, the maximum dimension D of array assembly 70 perpendicular to a centerline L is 2 mm or less. In a still even more preferred embodiment, array assembly 70 is sized and shaped for use with a 3 to 6 French catheter, has at least 48 elements 150, and each element has a width of 100 micrometers or less and a maximum length of 1.6 millimeters or less. Nonetheless, in other embodiments of the present invention differently sized and shaped array assemblies are envisioned.

Referring generally to FIGS. 1-5, one mode of operating system 20 is next described. Using a standard catheterization procedure, catheter 52 is inserted through opening O into the vasculature of body B and directed through the circulatory system into heart H. This procedure can include utilization of a guide wire that is then subsequently removed. The distal end 52b of catheter 52 is positioned along a desired region or site in heart H.

After placement of catheter 52, device 60 is inserted through port device 54 into a lumen of catheter 52 and is slidingly advanced towards distal end 52b. Advancement of distal end portion 60b continues in this manner until assembly 70 emerges through distal end 52b of catheter 52 and reaches a desired position within heart H as shown in FIG. 1. For materials used in equipment 50 or device 60 that are transparent or translucent to a selected imaging technique (such as polymeric resins that are generally transparent to x-ray based imaging), a marker that is opaque to such imaging technique can be included in catheter 52 and/or assembly 70 to aid with visualization.

After positioning in this manner, array 150a of device 60 is controllably activated with station 30 by sending corresponding electrical stimulus signals to generate ultrasonic energy with elements 150. The generated ultrasound is returned by the surrounding tissue and detected with elements 150. This detected ultrasound response is converted by elements 150 into corresponding electrical response signals that are transmitted to station 30 for processing. In one preferred approach, device 60 is operated in a standard, linear phased array mode, with elements 150 defining a side-looking aperture. In other embodiments one or more different modes can be utilized. Typically, circuitry 42 includes means to change phase and/or magnitude relationships of ultrasonic waveforms generated with elements 150 to implement standard ultrasonic imaging procedures. Subsystem 40 generates image data as a function of the stimulus and response signals from elements 150 for selective display with device 34 under control of operator input with devices 32 in a standard manner. In one embodiment, the activation stimulus for each element 150 is about 10 Megahertz (MHz). In other embodiments, the frequency is selected from a range of 3-15 MHz. In still other embodiments, one or more different frequencies or multiple frequency ranges could be utilized.

In further embodiments directed to navigation through the circulatory system and/or other body passageways, device 60 can be arranged with a longitudinal channel or passage to receive a guide wire. Guide wire placement is typically performed in advance of catheter 52. With an appropriate guide wire passageway, device 60 can be slidably advanced along a previously placed guide wire with or without utilization of catheter 52. Alternatively or additionally, device 60 can be of a self-directing, steerable variety that does not require a catheter or guide wire to navigate body passageways to a target site within the patient. In still other embodiments, device 60 can further include one or more elements to perform tissue ablation, such as might be desired for the treatment of atrial fibrillation. Such ablation elements may be structured to deliver energy in any standard modality including, but not limited to, microwave, laser, thermal conduction, ultrasound, and/or radio frequency energies. Alternatively or additionally, an inflatable balloon, stent delivery device, or such other medical diagnostic or therapeutic configuration commonly delivered through the circulatory system can be combined with the ultrasonic element array 150, adapting assembly 70 accordingly.

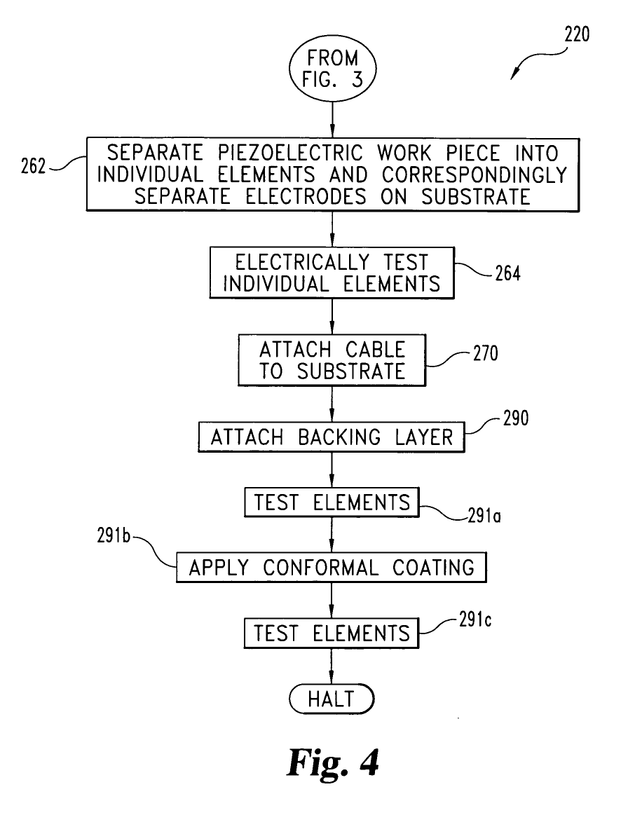

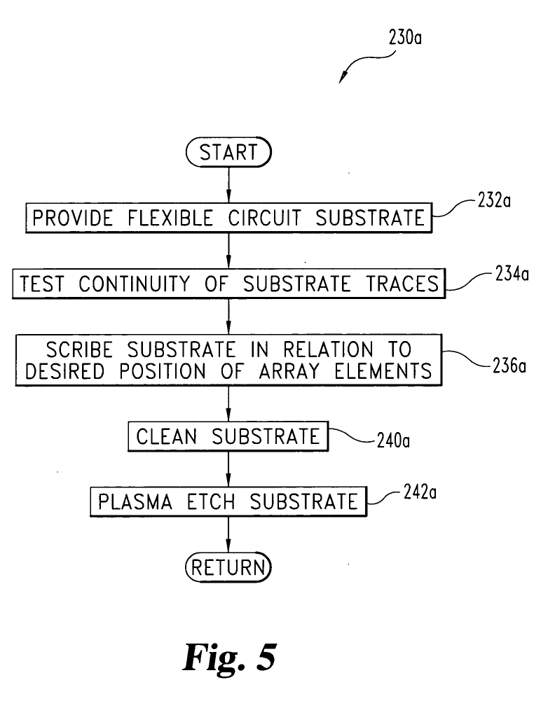

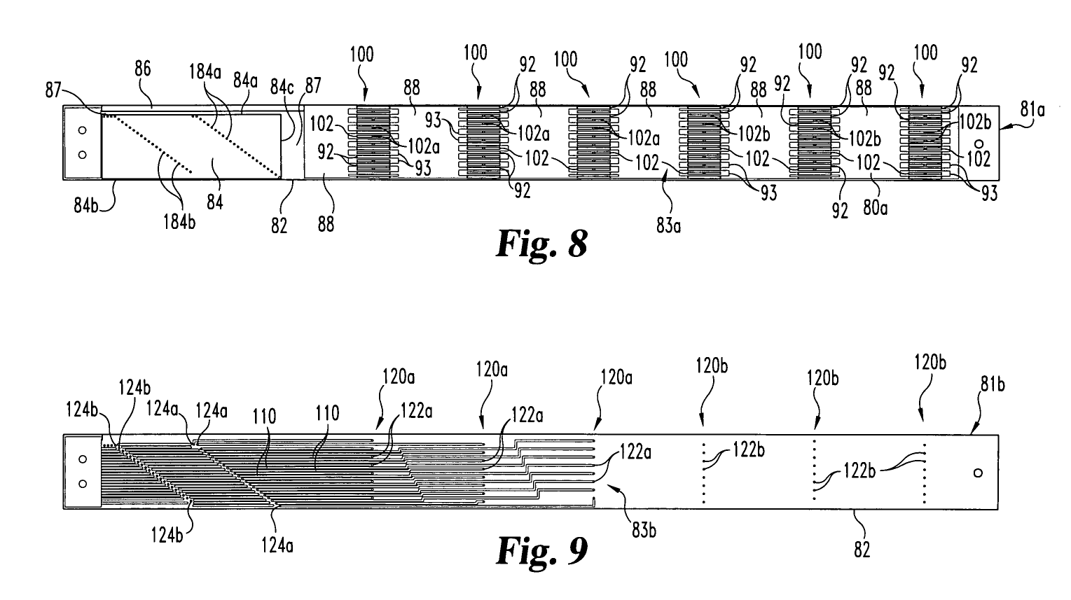

Turning next to the flowchart of FIGS. 3-4, one procedure for the manufacture of device 60 is described as process 220. Process 220 begins with the preparation of parts in operation 230. Referring also to the flowchart of FIG. 5, procedure 230a is described in which substrate 80 is prepared in accordance with operation 230. Procedure 230a begins with the provision of substrate 80. Substrate 80 is assembled from three flexible circuit substrate layers 81a, 81b, and 81c as illustrated in FIGS. 8-10, respectively. Layers 81a, 81b, and 81c (collectively layers 81) each include an electrically nonconductive base sheet 82 comprised of a flexible material, such as an organic polymer, just to name one example. Sheet 82 of each layer 81a, 81b, and 81c carries a different pattern 83a, 83b, 83c of electrically conductive traces and/or pads, respectively.

FIG. 8 illustrates pattern 83a of layer 81a. The illustrated view of layer 81a corresponds to side 80a of substrate 80 when assembled to which elements 150 are to be mounted as shown in FIG. 2. Pattern 83a includes mounting pad 84 electrically isolated at edges 84a and edge 84c from ground pad 86 by insulating region 87. Mounting pad 84 includes a number of electrically conductive through hole via contacts 184a and 184b that each correspond to a future placement of a different one of elements 150. Via contacts 184a and 184b are schematically represented by small circles in FIG. 8. Pattern 83a also includes grounding plane region 88 electrically connected to ground pad 86. Region 88 electrically interconnects ground pads 92 and defines apertures 93 therebetween in each of six cable mounting pad sets 100. In one form, region 88 is provided as a metallization layer or electrically conductive composite material through which apertures 93 are defined. Each pad set 100 includes ten ground pads 92 and eight signal pads 102. Each signal pad 102 is electrically isolated from grounding plane region 88 and pads 92 by being spaced apart therefrom within a corresponding aperture 93. As a result, each of pads 102 is surrounded by corresponding pairs of ground pads 92 and region 88 to provide ring-grounding for electronic signal noise reduction. Pads 102 each are electrically connected to an electrically conductive through-hole via contact 102a or 102b. Via contacts 102a and 102b are schematically represented by small squares in the center of each pad 102 in FIG. 8. Only a few individual pads 92, apertures 93, contacts 184a and 184b, contacts 102a and 102b, and pads 102 are designated by reference numerals to preserve clarity.

FIG. 9 illustrates pattern 83b of layer 81b. Pattern 83b includes traces 110 connected to electrically conductive through-hole via contact sets 120a. Sets 120a are each comprised of eight signal via contacts 122a. Traces 110 are also electrically connected to corresponding element via contacts 124a. Pattern 83b also includes electrically conductive through-hole signal contact vias 122b grouped as via sets 120b, and electrically conductive through-hole element contact vias 124b. Only a few of traces 110, contacts 122a, contacts 124a, vias 122b, and vias 124b are designated by reference numerals to preserve clarity.

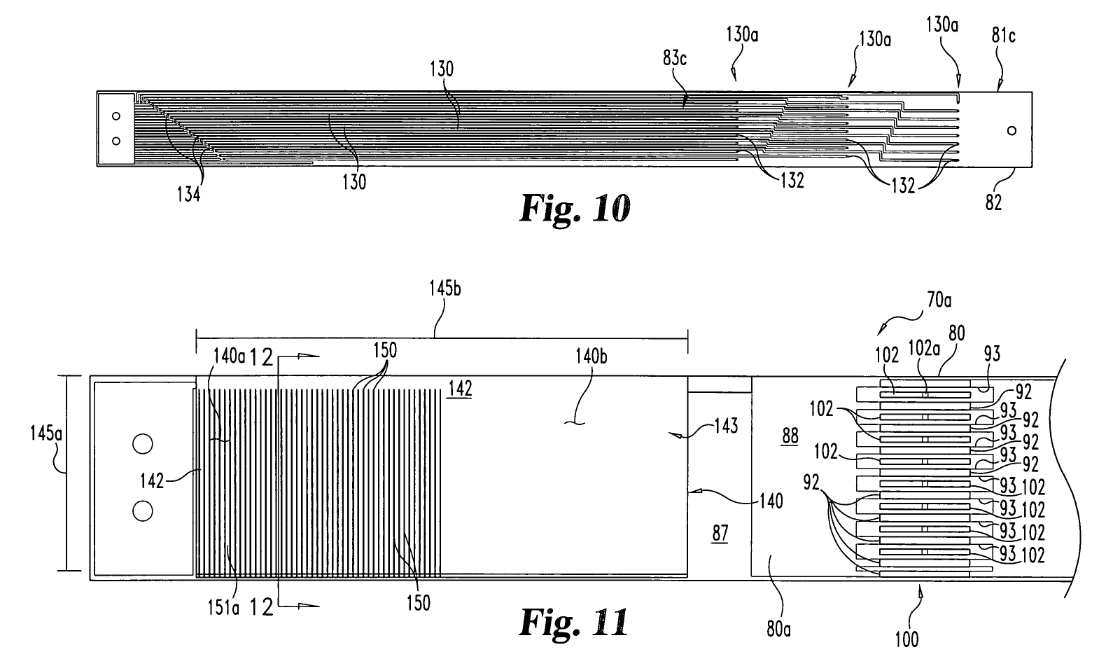

FIG. 10 illustrates pattern 83c of layer 81c. Pattern 83c includes electrically conductive traces 130 connected to electrically conductive through-hole via contact sets 130a. Sets 130a are each comprised of eight signal via contacts 132. Traces 130 are electrically connected to corresponding element contact vias 134. Only a few of traces 130, via contacts 132, and vias 134 are designated by references numerals to preserve clarity.

To assemble substrate 80, layers 81a, 81b, and 81c are lamninated together in a standard manner, with layer 81b being positioned between layers 81a and 81c. With regard to the electrical interconnections that result from lamination, each via contact 122a of a set 120a makes electrical contact with a corresponding through-hole via contact 102a; and each via contact 124a makes electrical contact with a different via contact 184a. As a result, traces 110 electrically interconnect via contacts 184a to via contacts 102a and the corresponding signal pads 102 of sets 100. The lamination of layers 81a and 81b together also provides electrical contact between via contacts 102b and contact vias 122b for corresponding signal pads 102. Further, through-hole contact vias 124b are placed in electrical contact with via contacts 184b of pad 84. The lamination of layer 81c to the bottom side of layer 81b places via contacts 134 in electrical contact with vias 124b, and corresponding via contacts 84b of pad 84. Traces 130 also electrically connect via contacts 132 to vias 122b, and corresponding via contacts 102b to electrically couple to corresponding signal pads 102. In this manner, the three right most sets 100 illustrated in FIG. 8 are electrically interconnected to pad 84 by corresponding via contacts 184b of layer 81a, vias 122b and 124b of layer 81b, and traces 130 of layer 81c; and the three left most sets 100 are electrically interconnected to pad 84 by via contacts 184a of layer 81a and traces 110 of layer 81b. Accordingly, substrate 80 electrically interconnects each signal pad 102 to a different element via contact 184a or 184b.

Pads 84, 86, 92, and 102 are provided in the form of exposed metallization. Typically, this metallization is the same as any used for region 88 and includes a metal such as nickel, copper, gold, platinum, silver, a combination of these, or other alloy thereof, that is plated and/or tinned to facilitate soldering. Generally any pattern 83a on side 80a is otherwise covered by an electrically nonconductive material. This material is typically in the form of a film or coating of a translucent or transparent polymeric resin, but can be comprised of one or more different materials as would occur to those skilled in the art. Any exposed conductors of side 80b of substrate 80 are also typically covered by such an electrically nonconductive material. Alternatively, some or all of such patterns may not be covered by an insulating material at this stage.

Returning to FIG. 8, procedure 230a continues with operation 234a. In operation 234a, electrically continuity of the substrate traces is tested in a standard manner. After this testing, procedure 230a proceeds to operation 236a. In operation 236a, pad 84 is scribed with a dicing saw through the corresponding metallization utilizing an alignment trace (not shown). Cuts are made generally parallel to one another and edge 84c of pad 84. Cuts are made for each of elements 150, and are generally located in the longitudinal center of the footprint to be occupied by each corresponding element 150 in assembly 70. From operation 236a, procedure 230a continues with operation 240a in which substrate 80 is cleaned. In one form, this cleaning includes a methanol alcohol wipe. In operation 242a, component preparation concludes with a two to three minute treatment of substrate 80 in a plasma-etching device. Procedure 230a then returns to process 220.

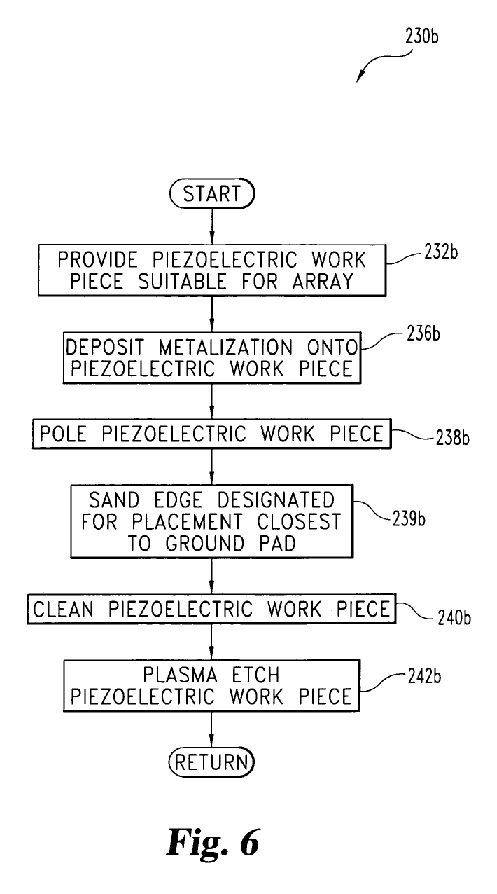

Referring to the flowchart of FIG. 6, procedure 230b to prepare a piezoelectric component in accordance with operation 230 of process 220 is described. Procedure 230b begins with operation 232b. In operation 232a, a piezoelectric work piece is provided that is suitable for division into the elements 150 of array 150a. In one form the piezoelectric material comprising the work piece includes CTS 3203HD; however, other material types can be utilized, including various other piezoelectric ceramics, composites, single crystals, and/or polymers with desired properties.

The work piece for array 150a is generally shaped in the form of a parallelepiped block of piezoelectric material. The work piece includes two opposing faces sized and shaped generally the same as pad 84 of substrate 80 described in connection with FIG. 8. One of these faces is mounted to pad 84 in a subsequent operation of process 120 leaving the other exposed. FIGS. 11 and 12 illustrate partial assembly 70a corresponding to later operations of process 220. In partial assembly 70a, the piezoelectric work piece is mounted, being designated as mounted work piece 140. The two opposing faces are designated by reference numerals 151a and 151b, respectively, in relation to mounted work piece 140.

In operation 236b of procedure 230b, metallization is deposited on the opposing faces of the work piece to provide electrodes. FIG. 12 designates the resulting electrodes by reference numerals 152a and 152b in correspondence to faces 151a and 151b. In one form, the electrode metallization includes low temperature sputtering of gold or an alloy thereof; however, other deposition processes and/or materials suitable for electrode formation can be utilized in different embodiments.

Procedure 230b continues with operation 238b in which the piezoelectric material is poled (polarized). Polarization is provided by subjecting the work piece to: (a) a slow ramp-up to an elevated temperature, (b) a slow ramp-up of a polarizing electric field (voltage) across the electrodes while maintaining the elevated temperature, (c) a slow ramp-down to room temperature while the field is maintained, and (d) a slow ramp down of the electric field while at room temperature. Temperature changes are performed at a rate of about 1 degree C per minute and voltage changes are gradual to a maximum of about 50-80 volts per mil thickness of material with a dwell time at maximum temperature and voltage of about 5 minutes. Performance parameters of the work piece are tested after poling. After parameter testing, procedure 230b continues with operation 239b in which an edge of the work piece is sanded that is designated for placement at pad edge 84a next to ground pad 86 (see FIG. 8) and then the work piece is cleaned in operation 240b with isopropyl alcohol and an ultrasonic cleaner. The work piece is also etched in a plasma-etching device in operation 242b. Procedure 230b then returns to process 220.

Returning to FIGS. 3 and 4, operation 230 of process 220 typically includes the preparation of other components to be described hereinafter. It should be understood that preparation of different components can typically be performed serially or in parallel and/or through an assembly-line or batch process. Process 220 proceeds from operation 230 to operation 250. In operation 250, the piezoelectric work piece is aligned for bonding to pad 84 by performing a visual inspection with a magnifying eyepiece to check position relative to features of side 80a of substrate 80. The piezoelectric work piece is bonded to pad 84 of substrate 80 using high-strength adhesive and a 30 pound clamp. Teflon fixtures on sides 80a and 80b of substrate 80 and the top of the piezoelectric work piece are used for the compression during bonding. Scribing of pad 84 in operation 236a of procedure 230a provides additional adhesive purchase for the secure bonding of each of elements 150 to substrate 80 as later formed from the work piece in a subsequent operation. Referring additionally to partial assembly 70a of FIG. 12, a portion 140a of mounted work piece 140 is illustrated. While the adhesive is not shown in FIG. 12 to preserve clarity, it is of a type that does not undesirably impede electrical connection between electrode 152b and pad 84, such as an epoxy with suspended carbon or metal particles, to name just one example, and/or may be of such a small thickness that it does not undesirably impede electrical coupling. In other embodiments, a different adhesive or nonadhesive-based procedure can be used to couple the piezoelectric work piece to substrate 80.

From operation 250, process 220 continues with operation 252 in which the electrical connection of mounted work piece 140 is tested. After this testing, an electrically nonconductive bead of epoxy adhesive is place along the edge that was sanded in operation 234b of procedure 230b in region 87 between pads 84 and 86. The deposited epoxy bead is designated as nonconductive support member 144 in the sectional view of FIG. 12. Member 144 is provided to reduce the likelihood of shorting between mounted work piece 140 and ground pad 86, and further provides a smooth transitional supporting structure between electrode 152a and ground pad 86 for formation of an electrical connection in a subsequent operation.

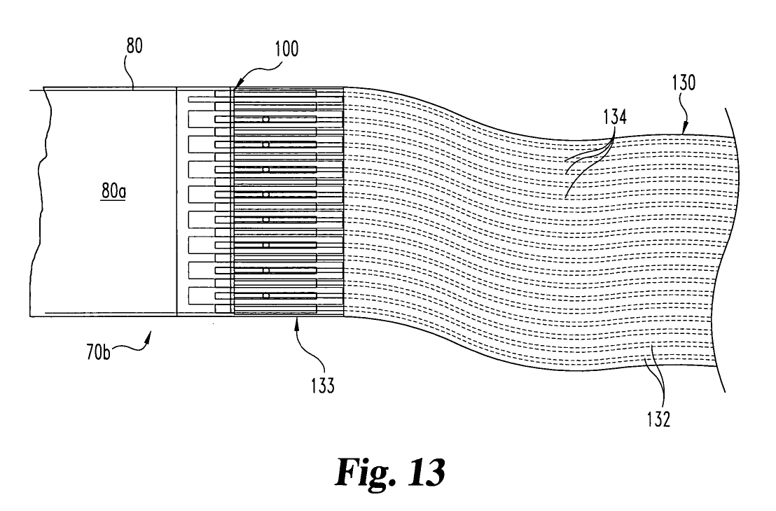

After member 144 has cured, the incomplete assembly is selectively masked, leaving only electrode 152a of face 151a, member 144, and ground pad 86 exposed in operation 256. The exposed area after masking corresponds to the rectangular region indicated by line segments 145a and 145b in relation to partial assembly 70a of FIG. 11; however, it should be understood that partial assembly 70a otherwise corresponds to a more advanced stage of manufacture. After masking in this manner, a layer of metallization 142 is deposited on the exposed region as illustrated in FIGS. 13 and 14. Accordingly, electrode 152a is electrically connected to ground pad 86 by metallization 142. In one form, this layer is formed by sputtering gold or an alloy thereof. In other forms a different electrically conductive material and/or deposition procedure can be utilized.

In operation 258, electrical connections are tested to verify proper electrical connectivity and isolation, as appropriate. Also, impedance is measured to verify proper electrical connection through the piezoelectric material of mounted work piece 140. From operation 258, process 220 continues with operation 261a. In operation 261a, matching layer stack 160 (see FIG. 12) is prepared from two different layers 160a and 160b having different acoustic properties selected to provide a desired acoustic interface with elements 150. Referring to FIG. 12, layers 160a and 160b are shown in relation to a corresponding acoustic matching member 160 later formed from the matching layer stack. The matching layer stack is prepared by lapping to different epoxies to the desired thickness and bonding them together to form the matching layer stack with a footprint the same as body 143. In operation 261b, the matching layer stack is bonded to the top of work piece 140 as shown in FIG. 12. Again, adhesive layers are not illustrated to preserve clarity.

In operation 262, mounted work piece 140 is divided into elements 150 each with a member 160. In one form, separation of elements 150 and layers 160a and 160b is performed with a dicing saw. The dicing saw is aligned relative to the assembly using an alignment trace (not shown) on side 80a of substrate 80 (the extra trace is used to put the blade in the proper plane, and give the location for the first cut), and then used to cut the mounted work piece 140 into 48 equally sized elements 150. The blade of the saw cuts through layer 160a, layer 160b, metallization 142, piezoelectric body 143, at least a portion of member 144, electrodes 152a and 152b, bonding adhesive, pad 84, and at least 5 micrometers into substrate 80 to ensure complete electrical separation of elements 150 from one another and separation of pad 84 into corresponding pieces that are electrically isolated from one another. After separation, each element 150 includes a portion of electrode 151a electrically connected to metallization 142 and a portion of electrode 151b connected to a corresponding portion of pad 84 and via 124. Each of via contacts 184a and 184b is sized and positioned to provide electrically isolated interconnection to a different one of signal pads 102 via traces 110 or 130 after performance of operation 262.

The partial assembly of FIGS. 11 and 12, corresponds to operation 262 after it has started, but before it is complete. As a result, a number of elements 150 have been separated in region 140a of FIG. 11, and separation of mounted work piece 140 into corresponding elements 150 has not been performed in region 140b. It should be appreciated that in other embodiments, a different separation technique (such as a laser cutting or selective etching to name just a few) may be alternatively or additionally utilized and/or some or all elements may be separated at substantially the same time, such that a partially separated state like that illustrated in FIGS. 11 and 12 would not typically result.

After operation 262, the assembly is tested in operation 264 to verify each of elements 150 is electrically connected to electrical ground at pads 86 and 92 through a portion of electrode 151a. Testing also verifies that each element 150 is electrically connected to a corresponding signal pad 102 through the electrical connection of corresponding portions of electrode 151b and pad 84, and that signal pads 102 remain electrically isolated from each other and electrical ground. In this manner, layer 81a predominantly defines traces for electrical ground and layers 81b and 81c predominantly define signal pathways.

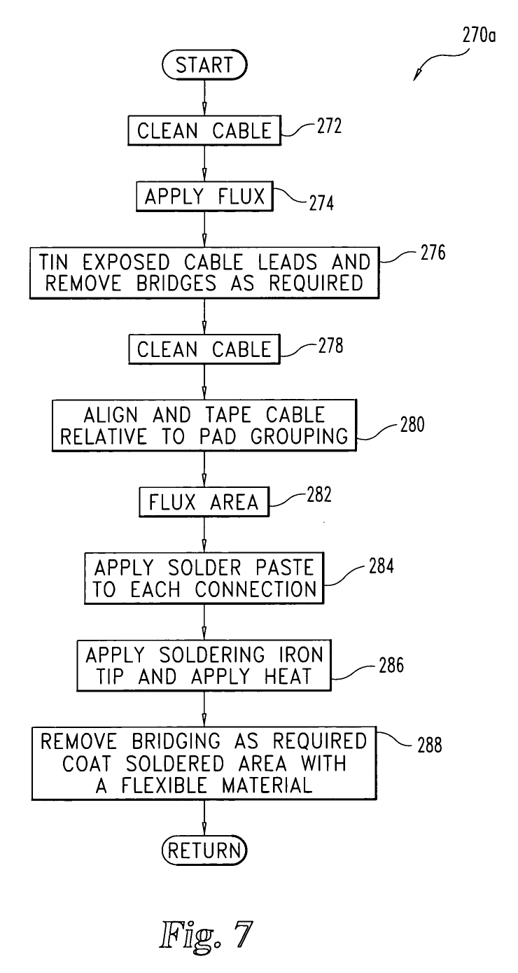

From operation 264 (FIG. 6), process 220 continues with cabling operation 270 (FIG. 7). In operation 270, six multiple conductor cables 130 (FIG. 15) are connected to substrate 80 to provide cabling 162 (FIG. 1). Each cable 130 is coupled to a corresponding one of pad sets 100 in accordance with procedure 270a described in connection with the flowchart of FIG. 7 and the partial assembly view of FIG. 13. Cable 130 includes eighteen conductors 132 terminating in a connection window region 133 where eighteen exposed contacts 134 are provided for connection to the eighteen pads (eight signal pads 102 and ten ground pads 92) of the corresponding one of pad sets 100. In one form, cable 130 is provided by W. L. Gore, and includes conductors 132 in the form of eighteen 48 gauge wires fixed in a spaced apart relationship to one another between two sheets of an organic polymer.

In operation 272 of procedure 270a, region 133 is cleaned with isopropyl alcohol. In operation 274, flux is applied to contacts 134. In operation 276, contacts 134 are tinned by a rapidly dipping region 133 in a solder pot of molten Sn60Pb40 solder with a dwell time of less than one second. The solder pot temperature is maintained just a few degrees above the melting point for Sn60Pb40 solder. In other embodiments a different tinning and/or plating procedure can be utilized to accommodate the cable connection operation, or may be absent. After tinning, solder bridges between contacts 134 are removed with a heated small diameter soldering iron tip. Region 133 is cleaned in operation 278. In operation 280, cable 130 is aligned with substrate 80 and taped to substrate 80, registering each of contacts 134 with a respective pad 92 or 102 of the corresponding pad set 100 to which it is to be connected. In operation 282, flux is applied to region 133 and corresponding pads 92 and 102. Solder paste (SN62, less than 25 micrometer ball size) is then applied by placing 5-10 individual balls to each contact 134 and matching pad 92 or 102 in operation 284. In operation 286, a soldering iron is placed on the contact area that has a tip shaped to contact the matched contacts 134 and corresponding pad set 100 simultaneously. After placement, the soldering iron applies heat for about 2 seconds, and then it is turned off. Accordingly, contacts 134 of cable 130 are each soldered to a respective pad 92 or pad 102 of the given pad set 100. Any bridging is removed in operation 288 if required, and a flexible potting material is used to coat each soldered region. Procedure 270a is performed for each of the pad set 100/cable 130 connections in operation 270 of process 220.

After performance of operation 270 to connect all six multiconductor cables 130. Next, in operation 290, acoustic backing layer 170 is bonded to side 80b of substrate 80. Side 80b is opposite side 80a. Backing layer 170 is formed of material selected to provide a desired acoustic/ultrasonic absorbing or damping property to broaden bandwidth and to reduce, if not eliminate, undesired acoustic/ultrasonic reflection during the operation of system 20. In one nonlimiting example, layer 160a is selected from a material having an acoustic impedance of about 2.1 to 4 megarayls (MRayls), layer 160b is selected from a material having an acoustic impedance of about 6-12 MRayls, and backing layer 170 is selected from a material having an acoustic impedance of about 3-5 MRayls. However, in other embodiments different acoustic stacks and arrangements may be utilized as would occurred to those skilled in the art.

After operation 290, proper electrical interconnection to elements 150 is tested in operation 291a. After testing, a conformal coating is applied to the array assembly area in operation 291b. In one nonlimiting example, a 5 to 8 micrometer parylene-c dimer material is used to provide a parylene coated array. This coating is not shown in the figures to preserve clarity. After coating, the device is again tested in operation 291c by driving elements 150 with an appropriate electrical source to stimulate ultrasound generation. Process 220 then halts.

Many other embodiments of the present invention are envisioned. Indeed, different ways of shaping, filling, and the like can be used. In still other embodiments a different kind of noncylindrical shape of array 150a can be provided in lieu of the generally flat, planar form illustrated. Alternatively or additionally, other materials, shapes, sizes, and designs can be utilized in connection with a flexible circuit substrate comprised of one or more layers with direct coupling to electrical signal pads via cabling. In yet other embodiments, a process other than process 220 is used to manufacture device 60 of the illustrated embodiments or variations of such devices as described herein.

All publications, patents, and patent applications cited in this specification are herein incorporated by reference as if each individual publication, patent, or patent application were specifically and individually indicated to be incorporated by reference and set forth in its entirety herein. Any theory, mechanism of operation, proof, or finding stated herein is meant to further enhance understanding of the present invention and is not intended to make the present invention in any way dependent upon such theory, mechanism of operation, proof, or finding. While the invention has been illustrated and described in detail in the drawings and foregoing description, the same is to be considered as illustrative and not restrictive in character, it being understood that only the selected embodiments have been shown and described and that all changes, modifications, and equivalents of the inventions as defined herein or by the following claims are desired to be protected.

What is claimed is:

1. A method, comprising:
providing an array assembly including a circuit substrate, several piezoelectric elements each mounted on a first side of the substrate, the elements being spaced apart form each other and arranged in a noncylindrical pattern, and an acoustic backing layer mounted on a second side of the substrate opposite the first side, the backing layer being structured to reduce ultrasonic reflection;
positioning the array assembly along a desired region within a subject's body by movement through a circulatory system of the subject's body; and
while the array assembly is positioned along the desired region, ultrasonically interrogating an internal portion of the subject's body with the array assembly to generate an one or more images corresponding to the internal portion.

2. The method of claim 1, wherein said interrogating includes:
transmitting a plurality of signals between the array assembly and equipment outside the subject's body through cabling coupled to the array assembly and the equipment; and
displaying the one or more images with the equipment.

3. The method of claim 1, wherein the substrate is of a flexible circuit type, the cabling includes 24 or more signal conductors electrically insulated from one another, the conductors are each electrically connected to a different one a corresponding number of signal pads carried on the substrate, and the elements each being electrically coupled to a different one of the signal pads.

4. The method of claim 1, wherein the array further includes one or more acoustic matching layer members mounted to each of the elements.

5. The method of claim 1, wherein a maximum cross sectional dimension of the array assembly taken perpendicular to a longitudinal centerline of the array assembly is 4 millimeters or less.

6. The method of claim 1, wherein the desired region is inside a heart passageway of the subject's body, the internal portion includes heart tissue, and said interrogating is performed through a side-looking aperture defined by the elements operating in a phased array mode.

7. The method of claim 1, wherein the flexible circuit substrate includes at least two different levels each having an electrically conductive trace pattern and each one the levels is separated from another by an electrically insulating layer.

8. An apparatus, comprising:
an array including a substrate with a number of electrically conductive traces, several piezoelectric elements mounted to a first side of the substrate to each make electrical contact with a different one of the traces, and a backing layer mounted to a second side of the substrate opposite the first side, the backing layer being comprised of a material selected to broaden element bandwidth and to reduce undesired ultrasonic reflection; and
cabling including several electrical conductors each electrically insulated from one another and each electrically connected to a different one of the electrically conductive traces at a distal end portion of the cabling to correspondingly provide a different electrical signal pathway for each of the elements; and
wherein the array and cabling are structured to place the array at a desired intracardiac site through a circulatory system of a human subject while a proximal end portion of the cabling remains outside the human subject, the array having a maximum cross sectional dimension of 4 millimeters or less taken through the elements and perpendicular to a longitudinal centerline of the array and cabling assembled together.

9. The apparatus of claim 8, further comprising equipment coupled to the cabling, the equipment including one or more processors operable to transmit electrical stimulus signals to the elements through the cable and receive electrical response signals from the elements to generate an image.

10. The apparatus of claim 8, wherein the elements are coplanar and number at least 24.

11. The apparatus of claim 8, wherein the substrate includes at least two different levels of electrically conductive traces, the levels each being separated from another by an electrically insulating layer.

12. The apparatus of claim 8, wherein the elements number at least 24, the substrate is of a flexible circuit type, and the array further includes a plurality of acoustic matching members each mounted to a different one of the elements.

13. A method, comprising:
providing a piezoelectric work piece mounted to a first side of a substrate, the substrate carrying a number of electrically conductive traces;
dividing the piezoelectric work piece into 24 or more elements with each of the elements electrically coupled to a different one of the electrically conductive traces; and
attaching a backing layer to a second side of the substrate, the second side being opposite the first side, the backing layer being operable to reduce undesired acoustic reflection.

14. The method of claim 13, which includes attaching one or more acoustic matching layers to the piezoelectric work piece before said dividing.

15. The method of claim 13, which includes:
masking the piezoelectric work piece and the substrate to expose a surface of the piezoelectric work piece and an electrically conductive pad carried by the substrate; and
depositing an electrically conductive material after said masking to electrically couple the exposed surface of the piezoelectric work piece and the pad before said dividing.

16. The method of claim 13, which includes attaching cabling to the substrate, the cabling including 24 or more conductors each electrically insulated from one another and each electrically connected to a different one of the electrically conductive traces.

17. The method of claim of claim 16, wherein said attaching includes coupling a number of multiconductor flex print cables to corresponding electrically conductive pad sets carried on the substrate with solder.

18. The method of claim 13, wherein the elements number at least 48.

19. The method of claim 13, wherein said dividing is performed with a saw.

20. The method of claim 13, wherein the substrate is of a flexible circuit type and includes at least two different conductive trace levels each separated from another by an electrically insulating layer.

21. An apparatus, comprising: an elongate device having a proximal end portion and a distal end portion and including:
an array of 24 or more piezoelectric elements mounted to a substrate to electrically make contact with a corresponding one of a number of electrically conductive traces carried by the substrate, the array being located along the distal end portion; and
cabling including 24 or more electrical conductors each electrically insulated from one another and each electrically connected to a different one of the electrically conductive traces to correspondingly define an independently operable electrical signal pathway for each of the elements; and
wherein the device is structured to place the array at a desired intracardiac site through a circulatory system of a human subject while the proximal end portion remains outside the human subject.

22. The apparatus of claim 21, further comprising equipment coupled to the cabling, the equipment including one or more processors operable to transmit electrical stimulus signals to the elements through the cable and receive electrical response signals from the elements to generate an image.

23. The apparatus of claim 21, wherein the elements number at least 48 and the substrate includes at least three different levels of electrically conductive traces, the levels each being separated from another by an electrically insulating layer.

24. The apparatus of claim 21, wherein the substrate is of a flexible circuit type, and the array further includes a plurality of acoustic matching members each mounted to a different one of the elements and a backing layer mounted to a side of the substrate opposite the elements, the backing layer being comprised of a material to reduce acoustic reflection.

25. An apparatus, comprising: an ultrasonic array assembly extending along a longitudinal centerline including:
a substrate having a first side opposite a second side, the substrate carrying a number of electrically conductive traces;
several piezoelectric elements mounted on the first side of the substrate, the elements each making electrical contact with a different one of the traces, the elements being arranged in a noncylindrical pattern; and
wherein a maximum cross sectional dimension of the assembly taken perpendicular the longitudinal centerline is 4 millimeters or less.

26. The apparatus of claim 25, further comprising cabling coupled to the substrate, the cabling including a plurality of electrical conductors each electrically insulated from one another and each electrically connected to a different one of the electrically conductive traces to correspondingly define an independent electrical signal pathway for each of the elements.

27. The apparatus of claim 26, further comprising equipment coupled to the cabling, the equipment including one or more processors operable to transmit electrical stimulus signals to the elements through the cable and receive electrical response signals from the elements to generate an image.

28. The apparatus of claim 25, wherein the elements are coplanar and number at least 24.

29. The apparatus of claim 25, wherein the substrate includes at least two different levels of electrically conductive traces, the levels each being separated from another by an electrically insulating layer.

30. The apparatus of claim 25, wherein the array further includes a plurality of acoustic matching layer members each mounted to a different one of the elements and a backing layer mounted to a side of the substrate opposite the elements, the backing layer being comprised of a material to reduce acoustic reflection.

31. A method, comprising:
providing an array coupled to cabling, the array including a substrate with a first side opposite a second side, several piezoelectric elements each mounted to the first side of the substrate, and a backing layer mounted to the second side of the substrate operable to reduce undesired acoustic reflection;
positioning the array along a desired region within a subject's body by movement through a circulatory system, a proximal portion of the cabling being positioned outside the subject's body while the array assembly is positioned along the desired region; and
ultrasonically interrogating an internal portion of the subject's body with the array along the desired region to generate one or more images corresponding to the internal portion.

32. The method of claim 31, wherein said interrogating includes:
transmitting a plurality of signals between the array assembly and equipment coupled to the proximal portion of the cabling outside the subject's body; and
displaying the one or more images as a function of the signals.

33. The method of claim 31, wherein the substrate is of flexible circuit type, the cabling includes 24 or more signal conductors electrically insulated from one another, the conductors are each electrically connected to a different one a corresponding number of signal pads carried on the substrate, and the elements each being electrically coupled to a different one of the signal pads.

34. The method of claim 31, wherein the elements number 24 or more and the elements are coplanar.

35. The method of claim 31, wherein the elements are arranged in a noncylindrical pattern.

36. The method of claim 35, wherein a maximum cross sectional dimension of the array assembly taken perpendicular to a longitudinal centerline of the array assembly is 3 millimeters or less.

37. The method of claim 31, wherein the desired region is inside a heart passageway of the subject's body, the internal portion includes heart tissue, and said interrogating is performed through a side-looking aperture defined by the elements, the elements being arranged in a noncylindrical shape.

38. The method of claim 31, wherein the flexible circuit substrate includes at least two different levels each having an electrically conductive trace pattern, each one the levels is separated from another by an electrically insulating layer, and the cabling includes a number of multiple conductor flex print cables connected to the substrate.

39. The method of claim 31, wherein the assembly includes several acoustic matching members each mounted to a different one of the elements.

40. The method of claim 39, wherein the acoustic matching members each include at least two layers of different acoustic impedance to provide a desired acoustic stack in cooperation with the backing layer relative to each of the elements.

41. A method, comprising:
providing an array of at least 24 piezoelectric elements mounted to a substrate to electrically contact a corresponding one of a number of electrically conductive traces carried by the substrate, and cabling with a distal end portion coupled to the array;
positioning the array at a desired site within a subject's body by movement through a circulatory system;
transmitting a plurality of electric stimulus signals to the array at the desired site through a proximal end portion of the cabling outside the subject's body; and
generating ultrasonic energy with each of the elements in response to the electric stimulus signals, the cabling including 24 or more signal conductors electrically insulated from one another, the signal conductors each being electrically connected to a different one of the conductive traces.

42. The method of claim 41, which includes:
providing the electric stimulus signals with equipment coupled to the proximal end portion of the cabling;
detecting ultrasound with one or more of the elements to return a corresponding number of electric response signals to the equipment; and
generating one or more images with the equipment as a function of the stimulus signals and the response signals.

43. The method of claim 41, which includes performing medical treatment with the array.

44. The method of claim 41, wherein the desired location is inside a passageway of a heart the subject's body, and which includes imaging one or more regions of the heart with the device and the equipment.

45. The method of claim 44, wherein the stimulus signals are provided in frequency range of about 3-15 megahertz and a maximum cross section dimension of the array taken perpendicular to a longitudinal centerline is 4 millimeters.

46. The method of claim 41, wherein the substrate is of a flexible circuit type and the cabling includes a number of multiple conductor flex print cables, and the elements number at least 48.

47. The method of claim 41, wherein the array includes several acoustic matching members each mounted to a different one of the elements and a backing layer mounted on a side of the substrate opposite the elements, the backing layer being operable to reduce undesired acoustic reflection.

48. The method of claim 47, wherein the acoustic matching members each include at least two layers of different acoustic impedance, the elements are arranged to be generally coplanar with one another.
